(19) United States
(12) Patent Application Publication
(10) Pub. No.: US 2004/0014191 A1
(43) Pub. Date: Jan. 22, 2004

(54) Nitroreductase enzymes

(76) Inventors: Nigel Minton, Salisbury (GB); Gill Anlezark, Salisbury (GB); Thomas Vaughan, York (GB)

Correspondence Address: STERNE, KESSLER, GOLDSTEIN & FOX PLLC, 1100 NEW YORK AVENUE, N.W., WASHINGTON, DC, 20005, US

(21) Appl. No.: 10/364,397

(22) Filed: Feb. 12, 2003

Related U.S. Application Data
(63) Continuation-in-part of application No. 09/913,068, filed on Dec. 28, 2001, now abandoned.
Application No. 09/913,068 is a 371 of international application No. PCT/GB00/00431, filed on Feb. 10, 2000.

(30) Foreign Application Priority Data
Feb. 10, 1999 (GB) 9903019.9

(51) Int. Cl. C12N 9/06; C12P 13/00; C12P 21/02; C12N 5/06; C12N 1/21; C12N 15/86; C07H 21/04

(52) U.S. Cl. 435/191; 435/456; 435/69.1; 435/320.1; 435/325; 435/128; 435/252.3; 536/23.2

(57) ABSTRACT

Nitroreductases, and genes encoding the same, are provided that demonstrate preferential catalytic conversion of the alkylating agent CB1954 into its highly cytotoxic 4-hydroxylamine (4HX) derivative, this derivative demonstrating anticarcinoma properties. Accordingly, the catalytic activity of the nitroreductase enzymes of the present invention may be employed to achieve catalysis of CB1954 into its cytotoxic derivative in a site-directed manner, such as by Directed-Enzyme Prodrug Therapy (DEPT).

The present invention relates to polypeptides and proteins having nitroreductase activity, to DNA and genes encoding these nitroreductases and to methods of obtaining such enzymes, DNA and genes.

A number of cancer therapies are based upon or exploit the conversion of a non-toxic prodrug into a toxic derivative.

One example concerns the monofunctional alkylating agent CB1954, which exhibits extreme toxicity towards the Walker 256 rat carcinoma as a result of the presence of a DT-diaphorase enzyme (DTD) which reduces the 4-nitro group of CB1954 to give a highly cytotoxic 4-hydroxylamine (4HX) derivative. CB1954 does not have the same effect on human carcinomas because human cells lack this enzyme but would be effective against human tumours if an enzyme such as DTD were externally supplied, e.g. in a Directed-Enzyme Prodrug Therapy (DEPT). The rat DTD, however, has a relatively poor specific activity for CB1954. The E. coli B nitroreductase enzyme (NfnB) was isolated as a more effective alternative and is the subject of EP-A-0540263. It exhibits a higher specific activity for CB1954, compared with the rat enzyme and is, therefore, currently the preferred enzyme in anti-cancer DEPT strategies.

Whilst the known E. coli enzyme receives widespread attention from cancer biologists seeking to develop gene based DEPT strategies, it has a number of drawbacks. These mostly relate to its activity against the preferred prodrug, CB1954—it has a relatively high Kₘ and low K_{cat}, and converts CB1954 into equimolar amounts of a relatively innocuous 2-hydroxylamino derivative (2HX) in addition to the highly cytotoxic 4-hydroxylamino species (4HX).

In relation to this specific prodrug, it is hence desired to provide an alternative to the known E. coli enzyme.

Additionally, and more generally, analogues of CB1954 and prodrugs other than CB1954 are known and further such precursors of potential toxic agents may become the focus of future therapies. In relation to all of these it is desired to provide further enzymes capable of use in converting prodrugs into drugs, e.g. for clinical uses.

It is an object of the present invention to provide nitroreductase enzymes, in particular nitroreductase enzymes for converting CB1954 and analogues thereof into drugs. It is a further object of the present invention to provide DNA and genes encoding nitroreductases, which DNA and genes in particular are incorporated into pharmaceutical compositions for prodrug therapies.

The present invention is based upon the discovery, purification, gene sequencing and/or expression of nitroreductases in bacteria and other microorganisms with hitherto unknown properties in converting prodrugs such as CB1954 into toxic derivatives. These nitroreductases posses properties which alone or in combination offer potential improvements compared with the known enzymes in this technology. The nitroreductases of the invention may be divided into different families based upon such characteristics as activity, product spectrum and/or amino acid sequence, and each given nitroreductase may fall into more than one of these families.

The present invention provides, in a first aspect, a nitroreductase enzyme, characterised in that it preferentially reduces CB1954 to a product that is a cytotoxic 4-hydroxylamine (4HX) derivative.

The enzymes of this aspect of the present invention confer the advantage that the product they generate from CB1954 contains a greater proportion of the cytotoxic 4HX derivative then the non-cytotoxic 2-hydroxylamino derivative. In preferred embodiments of the invention, the product is substantially entirely the cytotoxic derivative. The enzymes may hence be more efficient that those of the art as the enzymes of the invention produce more cytotoxic product for a given amount of pro-drug.

The present invention further provides, in a second aspect, a nitroreductase enzyme, characterised in that it reduces a prodrug to a toxic derivative with a Kₘ of less 700 micromolar, wherein the prodrug is selected from CB1954 and analogues thereof or other bioreductive drugs (Denny et al, B. J. Cancer, 1996, 74, pp S32-S38). The enzymes of the second aspect of the invention offer an advantage over the known E. coli derived enzyme in that they have a lower Kₘ (Kₘ of E. coli NfnB for CB1954 is around 862 micromolar) and thus have a higher affinity for substrate. Twenty nitrogen mustard analogues of CB1954 are described by Friedlos et al (J Med Chem, 1997, 40, 1270-1275).

More preferably, the Kₘ of the enzymes of the second aspect of the invention is less than 300 micromolar.

In a third aspect, the present invention provides a nitroreductase enzyme characterised in that it reduces a prodrug to a toxic derivative with a K_{cat} of at least 8, wherein the prodrug is selected from CB1954 and analogues thereof.

The enzymes of this aspect of the invention offer an improvement over that of the art, specifically the E. coli enzyme, in that they have an improved K_{cat}—i.e a higher value than for E. coli NfnB indicating a higher turnover of substrate by the enzyme. In preferred embodiments of this aspect of the invention, the K_{cat} of the enzymes is at least 10.

In a fourth aspect of the invention, there is provided a nitroreductase enzyme characterised in that it reduces CB1954 to a toxic derivative, it reduces SN23862 to a toxic derivative, it can use NADH and/or NADPH as electron donor and in that it shares no more than 50% sequence identity with the E. coli NfnB sequence. Preferably, the sequence identity is about 25% or less, this sequence identity being measured using the MEGALIGN (registered trade mark) software.

It has already been discussed how the known E. coli nitroreductase is well characterised and is fully sequenced. The nitroreductases of the fourth aspect thus represent a class of enzymes having nitroreductase activity, or being nitroreductase-like, which nevertheless are so different in amino acid sequence from the E. coli enzyme as to represent a separate family of nitroreductases.

This aspect of the invention thus advantageously provides a further class of nitroreductase enzymes for use e.g. in prodrug therapies.

The invention still further provides, in a fifth aspect, a nitroreductase enzyme characterised in that it reduces CB1954 or an analogue thereof to a toxic derivative, in that it shares at least 50% sequence identity with the rat DTD sequence and in that it does not contain a domain that is the same as or corresponds to amino acids 51 to 82 of the rat DTD sequence.

Sequence identity is suitably measured in the same way as described above in relation to the fourth aspect.

To determine whether a given nitroreductase contains a domain that is the same as or corresponds to amino acids 51 to 82 of the rat DTD sequence, the amino acid sequence of the given nitroreductase and of the rat DTD sequence are aligned using a conventional sequence alignment program, such as MEGALIGN (registered trade mark) made by DNASTAR, Inc.

If the alignment program indicates that there are no amino acids in the given sequence that, following the algorhythm of the program, are held to correspond to those at positions 51-82 of the rat DTD sequence then it is concluded that the rat domain is lacking from the given sequence.

This aspect of the invention thus provides a further class of nitroreductase enzymes for conversion e.g. of prodrugs into drugs. A nitroreductase in this class may also be obtained by deleting amino acid residues that correspond to residues 51-82 of the rat DTD from a known mammalian enzyme.

The nitroreductases of the invention may also be NADPH dependant. This property further distinguishes some enzymes of the invention from the known E. coli enzyme and the rat DTD.

It has been found that enzymes having one or more of the properties described may be obtained from bacteria of the family Bacillus, in particular a Bacillus selected from B. amyloliquefaciens, B. subtilis, B. pumilis, B. lautus, B. thermoflavus, B. licheniformis and B. alkophilus. This finding is of surprise in that at least three nitroreductase enzymes have been found in some species, in particular B. subtilis, B. lautus and B. pumilis, and as nitroreductases having the advantageous properties of the invention have not hitherto been identified in these bacteria, the currently used nitroreductase being obtained from E. coli.

In specific embodiments of the invention described in more detail below, a nitroreductase has a sequence selected from SEQ ID Nos 2, 4, 6, 8, 10, 12, 14, 16, 17, 18, 19, 20, 21, 23, 25, 27 and 29.

It has further been found that nitroreductases according to the invention may fall into more than one aspects of the invention. It is hence preferred that a nitroreductase of the invention possesses the properties of at least two aspects of the invention, and more preferably at least three aspects of the invention.

A specific embodiment of the invention is a nitroreductase of SEQ ID NO: 2 obtained from B. amyloliquefaciens this enzyme converts CB1954 into substantially only the cytotoxic derivative, hence falling into the first aspect of the invention, but also has a Kₘ that is improved compared to the E. coli enzyme, hence falling also into the second aspect of the invention.

A further specific embodiment of the invention is a nitroreductase from B. subtilis, SEQ ID NO: 9. This enzyme has a better K_{cat} than the E. coli enzyme, its K_{cat} being about 15 compared with about 6 for the E. coli enzyme, and hence falls into the third aspect of the invention. Additionally, this enzyme falls into the fourth aspect of the invention in that it reduces both CB1954 and SN23862 but shares less than 30% sequence identity with the E. coli sequence. Another B. subtilis enzyme, SEQ ID NO: 11 is similarly in both the third and fourth aspects of the invention, having a K_{cat} of about 15.

From the examples set out below it will be apparent how the further specific embodiments of the invention fall into at least two and even three aspects of the invention.

The enzymes of the invention are of use in enzyme directed prodrug therapy. Accordingly, it is preferred that they are provided in purified form.

A sixth aspect of the invention provides a pharmaceutical composition comprising a nitroreductase enzyme according to any of the first to fifth aspects of the invention in combination with a pharmaceutically acceptable carrier.

As mentioned above, the nitroreductase of the invention are of use in therapies such as directed-enzyme prodrug therapies. In these therapies, it is required to deliver the nitroreductase to the target site. This delivery can be achieved by delivering the enzyme itself or by delivering a DNA or gene coding for the enzyme.

In an example of the enzyme of the invention in use, a pharmaceutical composition is designed for a directed-enzyme prodrug therapy, and comprises a pharmaceutically acceptable carrier and a compound for converting a prodrug into a drug, wherein a compound is composed of at least a nitroreductase according to any of the first to fifth aspects of the invention conjugated to a targeting moiety.

The targeting moiety can suitably comprise an antibody specific for a target cell. Alternatively, the targeting moiety is a moiety preferentially accumulated by or taken up by a target cell.

A further example of delivery of the enzyme of the invention is achieved in a gene therapy-based approach for targeting cancer cells, as described in WO 95/12678. As described by Knox R. J. et al, the basis of this further prodrug therapy is delivery of a drug susceptibility gene into target, usually tumour or cancer, cells. The gene encodes a nitroreductase that catalyses the conversion of a prodrug into a cytotoxic derivative. The nitroreductase itself is not toxic and cytotoxicity used to treat the tumour cells arises after administration of a prodrug which is converted into the cytotoxic form. A bystander effect may be observed as cytotoxic drug may diffuse into neighbouring cells.

Thus, in this gene-based therapy, the nitroreductase is expressed inside a cell, in contrast to other delivery systems in which, for example, the enzyme itself is delivered accompanied by a targeting moiety.

Targeting of gene-based therapies may be achieved by providing a virus or liposome with altered surface components so that the delivery vehicle is recognised by target cells. Typically, transcriptional elements are chosen so that the gene coding for the nitroreductase enzyme will be expressed in the target cells, and preferably substantially only in the target cells. A number of viral-based vectors are suitable for this delivery. Retro-viral based vectors typically infect replicating cells. Adenoviral vectors and lentiviral-vectors are also believed to be suitable.

This delivery technology has been demonstrated by Bridgewater et al (Eur J Cancer 31a, 236-2370,1995). A recombinant retrovirus encoding a nitroreductase was used to infect mammalian cells, it being observed that infected cells expressing the nitroreductase were killed by application of CB1954.

Accordingly, a further aspect of the invention provides the use of a DNA sequence coding for a nitroreductase of the invention in manufacture of a medicament for prodrug therapy.

The medicament may take the form of a viral vector, comprising a DNA encoding the nitroreductase of the invention operatively coupled to a promoter for expression of the DNA. The medicament may take the form of a mini-gene comprising a DNA operatively linked to a promoter for expression of the DNA, the mini-gene being suitable for inclusion or incorporation into a targeting vehicle such as a microparticle.

Thus, an embodiment of the invention provides a viral vector comprising a nucleotide sequence encoding a nitroreductase according to any of aspects 1 to 5 of the invention, which nitroreductase converts a prodrug into a cytotoxic drug, and also a kit comprising the viral vector and the prodrug, and also a method of treatment of tumours which comprises administering an effective amount of the viral vector together with an effective amount of the prodrug.

The preparation and administration of these viral vectors may be substantially as described in WO 95/12678, the contents of which is incorporated herein by reference. The present invention relates to providing nitroreductase enzymes and genes and DNA coding therefore. The uses of those enzymes and genes may be as set out in WO 95/12678.

A nitroreductase can also be delivered by putting a gene of the invention into a bacteria that selectively colonises tumours, such as a clostridial (Lemmon et al, Gene Therapy, 1997, 4, 791-796) or Salmonella species.

A further aspect of the invention provides an isolated DNA encoding a nitroreductase according to any of the first to fifth aspects of the invention. The DNAs of this further aspect of the invention, and also the DNAs incorporated into vectors of the invention, preferably comprise a sequence which is selected from SEQ ID NOs: 1, 3, 5, 7, 9, 11, 13, 15, 22, 24, 26 or 28, together with fragments, derivatives and analogs thereof retaining nitroreductase activity according to one of the first to fifth aspects of the invention. The fragments, derivatives and analogs are suitably selected from sequences which retain at least 70% identity with the specific embodiments of the invention, or preferably at least 90% identity and most preferably at least 95% identity.

The enzymes of the invention can also be obtained by purification from cell extracts and may also be obtained by recombinant expression of DNA. A still further aspect of the invention lies in a method of preparing a nitroreductase enzyme, comprising expressing a gene in a bacterial cell, wherein the gene codes for a nitroreductase enzyme of the invention.

In an example of the invention described below in more detail, the gene expressed is a Bacillus gene or is a gene obtained by substitution, deletion and/or addition of nucleotides in or to a Bacillus gene.

The invention also provides the use of a nitroreductase according to any of the aspects of the invention in manufacture of a medicament for anti-tumour therapy, and the use of a compound comprising a nitroreductase according to any aspect of the invention conjugated to a targeting moiety in manufacture of a medicament for anti-tumour therapy.

The invention is now illustrated by the following specific examples and in the accompanying sequence listing in which:

SEQ ID NO: 2 is a nitroreductase from B. amyloliquefaciens (coded for by SEQ ID NO: 1) and designated “Bam YrwO”;

SEQ ID NO: 4 is a nitroreductase from B. subtilis (coded for by SEQ ID NO: 3) and designated “Bs YwrO”;

SEQ ID NO: 6 is a nitroreductase from B. subtilis (coded for by SEQ ID NO: 5) and designated “YrkL”;

SEQ ID NO: 8 is a nitroreductase from B. subtilis (coded for by SEQ ID NO: 7) and designated “YdeQ”;

SEQ ID NO: 10 is a nitroreductase from B. subtilis (coded for by SEQ ID NO: 9) and designated “YdgI”;

SEQ ID NO: 12 is a nitroreductase from B. subtilis (coded for by SEQ ID NO: 11) and designated “YodC”;

SEQ ID NO: 14 is a nitroreductase from E. coli (coded for by SEQ ID NO: 13) and designated “YabF”

SEQ ID NO: 16 is a nitroreductase from E. coli (coded for by SEQ ID NO: 15) and designated “YheR”;

SEQ ID NO: 17 is a nitroreductase from H. influenzae;

SEQ ID NO: 18 is a nitroreductase from T. aquaticus;

SEQ ID NO: 19 is a nitroreductase from Synechocystis sp PCC 6803;

SEQ ID NO: 20 is a nitroreductase from A. fulgidus;

SEQ ID NO: 21 is a nitroreductase from A. fulgidus.

SEQ ID NO: 23 is a nitroreductase from Campylobacter jejuni (coded for by SEQ ID NO: 22);

SEQ ID NO: 25 is a nitroreductase from Porphyromonas gingivalis (coded for by SEQ ID NO: 24);

SEQ ID NO: 27 is a nitroreductase from Yersinia pestis (coded for by SEQ ID NO: 26); and

SEQ ID NO: 29 is a nitroreductase from Helicobacter pylori (coded for by SEQ ID NO: 28).

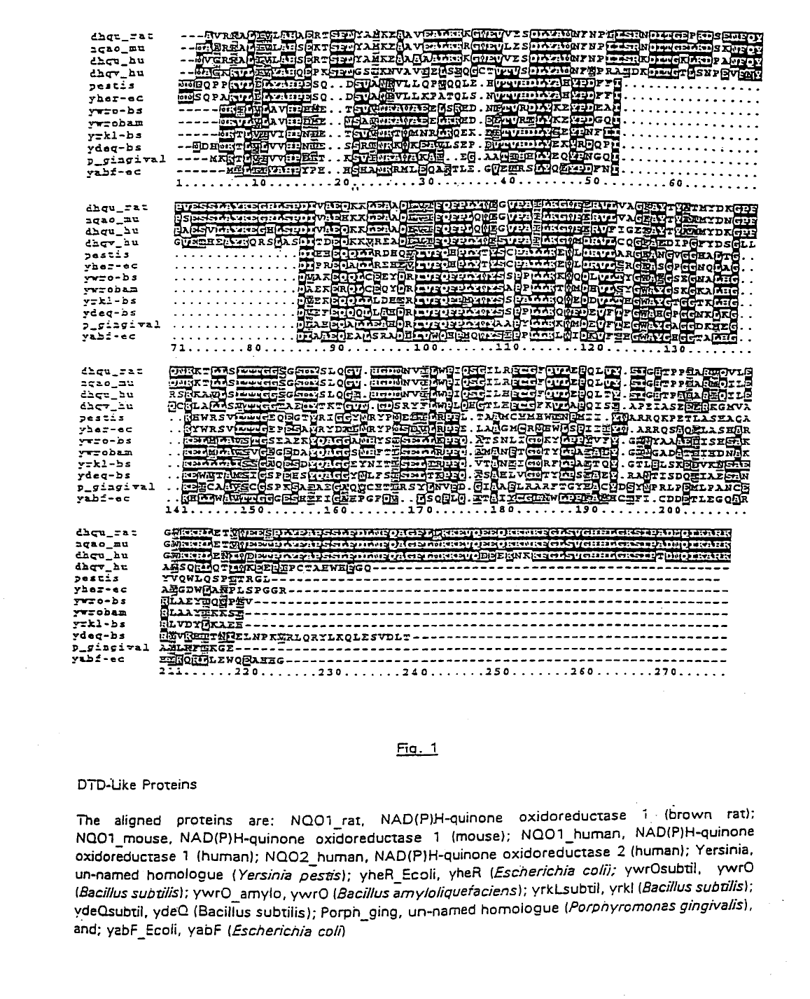

The invention is also illustrated by reference to the accompanying Tables and FIGS. 1 to 4, in which:—

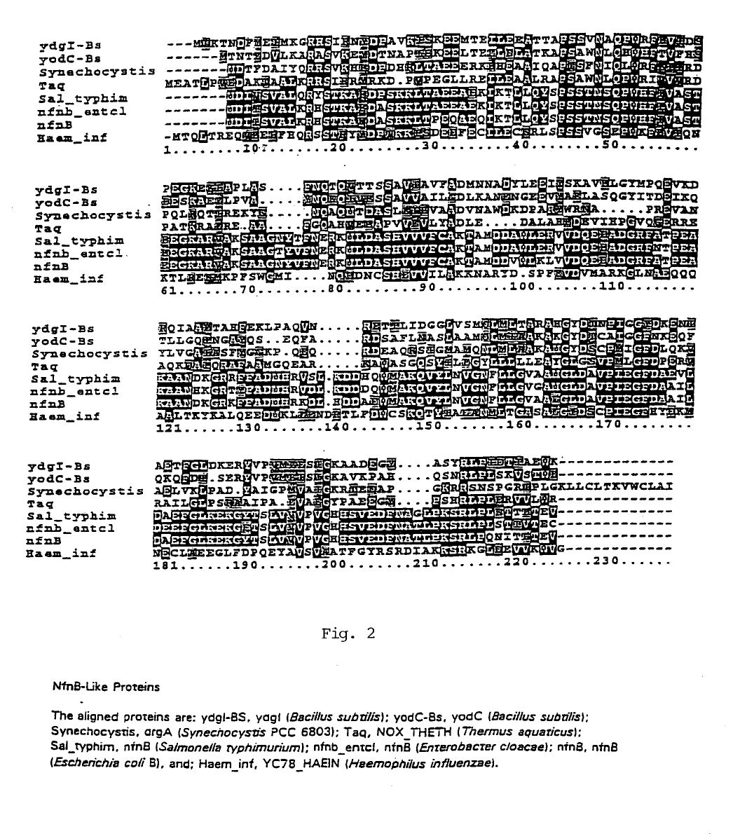

FIGS. 1 and 2 show sequence comparisons as set out in more detail in Example 8;

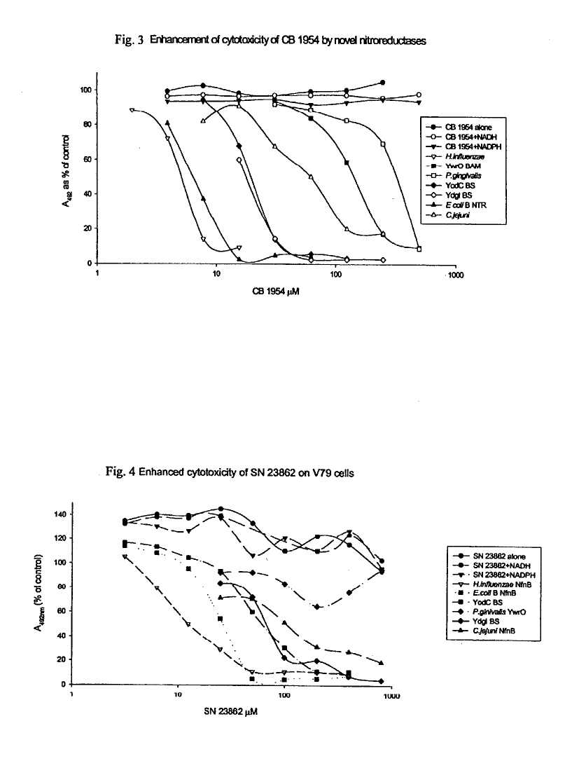

FIG. 3 shows enhancement of cytotoxicity of CB 1954 using enzymes of the invention; and

FIG. 4 shows enhanced toxicity of SN 23862 using enzymes of the invention

### EXAMPLE 1

A Nitroreductase Enzyme/Gene from Bacillus amyloliquefaciens

Briefly, extracts of Bacillus amyloliquefaciens were shown to possess nitroreductase activity. To purify this activity, crude cell extracts were subjected to ammonium sulphate, fractionation and anion exchange chromatography. The purified material was subject to N-terminal amino acid sequence analysis and the information obtained used to cloned the gene via a PCR-based strategy. Following determination of its nucleotide sequence the gene was overexpressed in E. coli and the resultant recombinant protein purified and characterised see table 1.

This analysis showed that the enzyme had properties which were distinct from that of E. coli NfnB. Thus the protein had a more favourable Kₘ for CB1954 (1.5-fold lower than the E. coli B NfnB) and furthermore converted CB1954 into the 4HX form alone. It also differed from the E. coli B NfnB in that the enzyme showed no activity against the prodrug SN23862.

The isolated enzyme/gene represents a significant improvement over the E. coli NfnB enzyme with respect to its activity against the prodrug CB1954 ie., it produces only the 4HX derivative and has an improved Kₘ for CB1954.

A comparison of the amino acid sequence of the isolated enzyme revealed that it shared a very low level of homology to the rat DTD (c. 25%), but exhibited high homology (70% sequence identity) with the predicted product of a gene that has been discovered in the Bacillus subtilis genome sequencing project, designated ywrO. On this basis, we have designated the cloned Bacillus amyloliquefaciens gene ywrO, and its encoded enzyme YwrO.

YwrO BAM is a tetrameric flavoprotein (monomeric molecular mass approximately 22.5 kDa by SDS-PAGE, native molecular mass approximately 90 kDa by gel filtration). Although it shares sequence homology with rat DTD it differs in its enzymic properties in that it can use only NADPH as cofactor (Kₘ 40 μM). In common with DTD it can reduce CB1954 but not SN23862, reduction of CB1954 resulting in formation of the 4HX product only (Kₘ 617 μM, k_{cat} 8.2). It shows a high affinity for the quinone menadione (Kₘ 3.4 μM) and has azoreductase and flavin reductase activity (Kₘ for FMN 53 μM, Kₘ for FAD 209 μM).

In more detail, N-terminal amino acid sequencing of the purified Bacillus amyloliquefaciens nitroreductase enzyme resulted in the following sequence, Met-Lys-Val-Leu-Val-Leu-Ala-Val-His-Pro-Asp-Met-Glu-Asn-Ser-Ala-Val-Asn. When this sequence was used to search available protein databases strong homology was noted with the predicted amino acid sequence of a hypothetical protein, YrkL, identified in the Bacillus subtilis genome sequencing project. Significant homology was also evident with two proteins, YabF and YheR, identified during the course of the determination of the Escherichia coli genome. These three hypothetical proteins shared weak homology with a number of mammalian quinone reductases and NAD(P)H-oxidoreductases, such as the rat DTD.

In view of this observation, a strategy was formulated whereby sequence homology between the identified bacterial proteins, together with the determined N-terminal amino acid sequence of the discovered Bacillus amyloliquefaciens enzyme, was used to amplify a region of the desired encoding gene from the Bacillus amyloliquefaciens genome. The one primer utilised in PCR was a degenerate oligonucleotide sequence which corresponded to a DNA sequence capable of coding for the N-terminal octa-peptide Val-His-Pro-Asp-Met-Glu-Asn. It was composed of the following nucleotides, 5′-GTNCAYCCNGATATGGARAA-3′, where Y indicates the presence of a T or C, R indicates the presence of A or G, and N indicates the presence of either T, C, G or A. The second primer was based on the hypothetical sequence His-Gly-Trp-Ala-Tyr-Gly which was found to be entirely conserved between the hypothetical bacterial proteins YrkL (Bacillus subtilis) and YabF (E. coli), and partially conserved in YheR (E. coli). The degenerate oligonucleotide mixture synthesised corresponded to the antisense DNA coding strand, viz., 5′-CCRTANGCCCANCCRTG-3′.

| | | |
|---|---|---|
| E. coli | YheR (90-95) | Arg Gly Phe Ala Ser Gly |
| E. coli | YabF (84-89) | His Gly Trp Ala Tyr Gly |
| B. subtilis | YrkL (85-90) | His Gly Trp Ala Tyr Gly |

The two primers were employed in PCR using chromosomal DNA isolated from Bacillus amyloliquefaciens and an amplified DNA fragment of the expected size (approximately 230 bp) obtained. This was cloned into plasmid pCR2.1 TOPO (Invitrogen) and its nucleotide sequence determined. Translation of the sequence obtained demonstrated the presence of an open reading frame which encoded a polypeptide which shared 66% sequence similarity with YrkL.

To obtain the entire structural gene, an approach was employed based on inverse PCR. In essence, B. amyloliquefaciens DNA was cleaved with the restriction enzyme StyI and the fragments generated circularised through their subsequent incubation with DNA ligase. The ligated DNA was then used as the template for a PCR employing two divergent primers based on the sequenced 220 bp fragment. These were BamNTR11 (5′-GCTTATTGACCGCTGAG-3′) and BamNTR14 (5′-GTACAGTGCGCCTCCGC-3′). A 2.9 kb fragment was generated, cloned into pCR2.1 TOPO (Invitrogen) and the sequence of the insert determined. This allowed the identification of the nucleotide sequence of the remaining parts of the B. amyloliquefaciens gene. Using this information, a contiguous copy of the entire structural gene was amplified from the B. amyloliquefaciens chromosome using primers which encompassed the translational start codon (5′-GGTGTGATACATATGAAAGTATTG-3′) and resided 3′ to the translational stop codon (5′-CGGGGATTCGAATTCTTTCTCAGG-3′). The primer at the 5′-end of the gene was designed such the sequence immediately 5′ to the ATG start codon became CAT. This change created an NdeI restriction site (CATATG), thereby allowing the cloning of the gene into the equivalent site of the expression vector pMTL1015. This manipulation facilitated the subsequent overexpression of the gene, as insertion of the gene at this point positions the start codon at an optimum distance from the vector borne ribosome binding site.

The strategy employed to clone the BM YwrO gene could be similarly employed to clone further genes encoding novel nitroreductases. This would involve purifying the desired enzyme activity from a cell lysate, and then determining the N-terminal sequence. The data obtained could then be used to design an oligonucleotide primer corresponding to the sense strand of the DNA encoding part or all of the determined amino acid sequence. This primer could then be used, in conjunction with a second primer, to amplify part of the gene encoding the nitroreductase from the chromosome of the bacterial host using PCR. The second primer would correspond to the antisense strand of an internal portion of the targeted gene. Its design would be based on regions of homology which are conserved amongst the type of nitroreductase family that is sought. Thus, in the case of the DTD-like family, the oligonucleotide would, for example be based on the conserved motif His-Gly-Trp-Ala-Tyr-Gly (ie., amino acid residues 85-90 in the BS YrkL protein). In the case of the NfnB-like family, the oligonucleotdie could be based on the motif Glu-Arg-Tyr-Val-Pro-Val-Met (ie., amino acid residues 170-176 in the BS YodC protein).

Such amplified fragments could then be cloned and sequenced, and new primers designed based on this sequence to isolate the flanking regions of the gene by PCR. Once these have been cloned and sequenced, the entire, contiguous structural gene may be amplified using primers which extend beyond the 5′ and 3′ end of the translational start and stop codons.

Cloning of genes encoding novel nitroreductases may also be achieved without recourse to N-terminal sequencing of the enzyme, or even its purification. This would involve basing the sequence of both of the oligonucleotides used in the initial PCR reaction on amino acid sequence motifs conserved amongst the two identified nitroreductase families. Thus, in the case of the NfnB-like family, a sense primer (eg., 5′-ATTTCTAAAGAAGAGCTGACGGAA-3′) based on the motif Ile-Ser-Lys-Glu-Glu-LeuI-Thr-Glu (ie., amino acid residues 13 to 20 of BS YodC) could be employed with an antisense primer (eg., 5′-CATTACCGGTACATAGCGTTC-3′) based on the sequence motif Glu-Arg-Tyr-Val-Pro-Val-Met (ie., amino acid residues 170 to 176). In the case of the DTD-family a sense primer (eg., 5′-CATCCGGATATGGAAAAT-3′) based on the motif His-Pro-Asp-Met-Glu-Asn (ie., amino acid residues to 9 to 14 of BM YwrO) could be employed with the an antisense primer (eg., 5′-TCCATATGCCCATCCATA-3′) based on the sequence motif Tyr-Gly-Trp-Ala-Tyr-Gly (ie., amino acid residues 85 to 90). Once amplified, the rest of the gene could be isolated using the same procedure as outlined above.

### EXAMPLE 2

Bacillus subtilis Nitroreductases

As indicated above in Example 1, comparative analysis of the B. subtilis genome sequence with the amino acid sequence of the isolated B. amyloliquefaciens enzyme demonstrated the existence of an enzyme (YwrO) which shared 70% sequence identity. Unexpectedly, B. subtilis was found to possess two homologues, YrkL and YdeQ, which share 54% and 51% sequence homology, respectively, with the B. amyloliquefaciens enzyme. All three enzymes share no homology with the E. coli B NfnB. They do, however, exhibit weak similarity (c. 25%) to the rat DT-Diaphorase (DTD). Whilst these proteins share a low level of sequence similarity to DTD, and other mammalian equivalents, they are characteristically smaller. This is because of the absence of an extensive internal protein domain at the N-terminus of the protein. Thus, the functional equivalent domain of the rat DTD between amino acid residues 51 to 82, are missing from the BM YwrO protein. In addition, the rat DTD has an extra COOH-terminal domain. These bacterial enzymes are thus distinct from their mammalian equivalents.

A further analysis of the B. subtilis genome, demonstrated that two homologues of the E. coli NfnB gene were present. Their encoded proteins (YdgI and YodC) share a barely detectable level of sequence conservation with EC NfnB, of around 20% sequence identity.

Bacillus subtilis was thus found to carry at least 5 different enzymes with nitroreductase activity. These are split into two families, thus;—

| | |
|---|---|
| DTD-like- | 3 members:- YwrO, YrkL, YdeQ |
| NfnB-like- | 2 members:- YdgI, YodC |

### EXAMPLE 3

Recombinant Production of Nitroreductases from Bacillus subtilis

The DNA encoding all 5 B. subtilis nitroreductase enzymes were cloned from genomic DNA using PCR and the resultant genes, following authentification by nucleotide sequencing, subcloned into a propriety CAMR expression vector (pMTL1015). The expression clones generated have been used to overproduce each of the 5 proteins and the enzymic activity of each assessed in crude lysates. This analysis has demonstrated that whilst the B. subtilis YwrO shares similar properties to the B. amyloliquefaciens homologue (ie., converts CB1954 to the 4HX derivative alone, but is inactive against SN23862), YrkL and YdeQ have no activity against either of the two prodrugs tested (CB1954 or SN23862) but they may be active against other prodrugs.

Despite the extremely limited sequence similarity to EC NfnB, YdgI and YodC are active against both CB1954 and SN23862. They do, however, produce both the 2HX and 4HX derivatives of CB1954. Their characterisation has shown that they turn over CB1954 at higher rates than EC NfnB (YodC k_{cat} 58, YdgI k_{cat} 30.3 cf 6 for NfnB). Both show a high affinity for menadione and flavins, but they differ in that whereas YdgI uses both NADH and NADPH, YodC shows a preference for the latter. The native molecular mass of YodC (approximately 90 kDa) indicates that it is tetrameric (molecular mass estimated from amino acid sequence and by SDS-PAGE being approximately 22 kDa) whereas YdgI appears to be a dimer in the native state (molecular mass by gel filtration approximately 49 kDa).

These finding are further illustrated in Table 2.

### EXAMPLE 4

Bacillus lautus & Bacillus pumilis Nitroreductases

From 103 soil sample isolates tested, two strains (Bacillus pumilis CP044 and Bacillus lautus CP060) had been previously chosen as possessing extracts which showed the most rapid reduction of both CB1954 and SN23862. Purification experiments demonstrated that the activity in both extracts was distributed across three distinct peaks. The presence of more than one enzyme activity is consistent with our discovery of multiple forms of proteins in Bacillus able to turnover prodrugs. Eventual purification of the three enzymes of B. pumilis CPO44 revealed that no one candidate exhibited properties which were an improvement on the E. coli NfnB enzyme. In contrast, the proteins in peak 1 and peak 3 of the B. lautus CP060 were determined to offer advantage over NfnB.

Thus, whilst the enzyme in peak 1 did not produce the required 4HX derivative of CB1954, it exhibited a 4-fold lower Kₘ with the prodrug SN23862. The enzyme of peak 3 was, however, deemed to be of greatest value as it converted CB1954 solely into the 4HX derivative and had a Kₘ approximately 4-fold lower than NfnB. Furthermore, it also had activity against SN23862. In this respect it shares the properties of both the Bacillus DTD-like family (ie., it produces only the 4HX derivative) and the NfnB-like family (ie., it is active against SN23862)—these findings are illustrated in Table 3.

### EXAMPLE 5

N-Terminal Sequencing of B. lautus Nitroreductase

Electrophoretic separation of the peak 3 demonstrated that 4 protein bands were present which could account for the observed prodrug activity. All four were subjected to N-terminal amino acid sequencing and the activity localised to the fourth protein band from which the nitroreductase may be purified.

### EXAMPLE 6

Detection of Nitroreductase Activity in Thermophile Extracts

As an alternative source novel enzymes, a preliminary screen of CAMRs thermophile collection was undertaken. Enzymes from this source may have the advantage of greater stability, and therefore longevity of action. Strains were selected on the basis either of sensitivity to CB1954, or those which are resistant but which impart a yellow/golden coloration to agar containing prodrug.

Two of these strains (B. thermoflavus and B. licheniformis) generated the cytotoxic 4HX form and were selected for further study.

### EXAMPLE 7

Identification of Further Nitroreductase Enzymes

Having identified the two families of nitroreductase in Bacillus, a search was undertaken of both finished and unfinished genomes for homologues, using YwrO and YodC/NfnB. On the basis of this search homologues of YwrO were identified in the genomes of Yersinia pestis and Porphyromonas gingivalis, and homologues of NfnB in the genomes of Pyrococcus furiosus, Haemophilus influenza, Synechocystis PCC 6803, Campylobacter jejuni, Archaeglobus, Helicobacter pylori, Heliocbacter fulgidus and Thermus aquaticus.

In addition to the above, two E. coli genes were found to be homologues of rat DTD and YwrO, and were designated Yher and YabF. They were discovered to share the characteristic of YwrO in that they lack the internal protein domain found in the rat DTD enzyme and functional mammalian homologues.

(i) P. gingivalis YwrO Homologue

P. gingivalis YwrO homologue is a dimeric flavoprotein with native molecular mass estimated by gel filtration at 40 kDa. Although it shares sequence homology with DTD and forms only the 4HX reduction product of CB1954 (Kₘ 1200 μM, k_{cat} 3.2), it differs from DTD in that it is active with SN23862 and it can only use NADH as cofactor (cf DTD which can use either NADH or NADPH and is inactive with SN23862). It can reduce azodyes but it is inactive with menadione or flavins.

(ii) C. jejuni NfnB Homologue

C. jejuni NfnB homologue produces only the 4HX reduction product of CB1954 (Kₘ 143 μM, k_{cat} 11.2) using NADPH as cofactor and it is also active with SN23862. It can use the quinone menadione as substrate as well as azodyes and the flavins FMN and FAD.

(iii) Archaeoglobus fulgidus NfnB Homologue

Archaeoglobus fulgidus NfnB homologue is a dimeric flavoprotein of 42 kDa native molecular mass, producing the 4HX derivative of CB1954 only (Kₘ 690 μM, k_{cat} 56.2) using NADPH as cofactor. It is also active with SN23862 and menadione (Kₘ 9 μM), but does not decolourise azodyes and has only weak flavin reductase activity.

(iv) H. influenzae and H. pylori NfnB Homologues

Both these enzymes are dimeric flavoproteins and form the 4HX reduction product of CB1954 using NADPH in preference to NADH, but have no activity with azodyes. The former also lacks activity with the quinone menadione and flavins FMN or FAD. Both however have weak activity with SN23862 and may be active with other prodrugs.

(v) Y. pestis nfnB Homologue and Synechocystis YwrO Homologue

Both these proteins reduce CB1954 but produce only the relatively non-toxic 2HX derivative using NADPH as cofactor. They do however show activity with SN23862 and the former can also reduce azodyes.

### EXAMPLE 8

Comparison of Nitroreductase Sequences

We compared the amino acid sequences of nitoreductases according to the invention with each other and with known rat, human and E. coli sequences, and the results are illustrated in FIGS. 1 and 2. In FIG. 1, rat, mouse and two human sequences make up the first four lanes for comparison purposes. It is evident that nitroreductases of the invention are lacking a sequence from positions 51-82 of the rat sequence.

In FIG. 2, sequences of nitroreductases of the invention are compared with the known E. coli sequence, which is designated nfmB in the second-to-last lane.

### EXAMPLE 9

Materials and Methods

Reagents

CB 1954 and SN 23862 were generous gifts from Dr D. Wilman, Institute of Cancer Research, Sutton, Surrey and Prof. W. Denny, University of Auckland, New Zealand, respectively. DMSO was obtained from Aldrich. Restriction endonucleases were from NBL and New England Biolabs. T4 DNA ligase was from Boehringer Mannheim, Taq polymerase from Bioline and native Pfu polymerase from Stratagene. DNA purification reagents were from Cambio (Gene Releaser™ resin), Promega (Wizard™ plasmid purification resin) and Qiagen (PCR clean-up kit and gel-extraction kit). PCR-BluntII-TOPO was obtained from Invitrogen. Deoxyribonucleotides were from Fermentas. Oligonucleotides were synthesized by CAMR Structural Sciences. DNA labelling reagents were from Amersham (ECL kit). All other reagents from Sigma Chemical Co.Ltd

Enzymes

E. coli B nitroreductase was purified as previously described (1). All other recombinant enzymes were purified by anion exchange chromatography except for the NfnB homologue of C. jejuni (for details see Results section).

Enzyme Assays

Quantitative assays using CB 1954 or SN 23862 as substrate were carried out at 37° C. by HPLC as previously described (2) (3). When qualitative assays were used to identify column fractions the standard conditions were as follows: 1 mM prodrug, 2 mM NAD(P)H, 4% DMSO in 100 mM sodium phosphate buffer pH 7, 37° C. Incubation times varied according to the enzyme activity being studied. Assays using menadione as substrate were carried out spectrophotometrically as previously described (1) using cytochrome c as terminal electron acceptor and similar procedures were used to assay flavin reductase activity with FMN and FAD as substrate and with cofactors NADH and/or NADPH. Azoreductase activity was assessed qualitatively by incubating aliquots of enzymes with 500 μl o-methyl red or p-methyl red (200 μM in 20 mM BisTris pH 7.0, 1% DMSO)+NADPH (500 μM) as cofactor at 37° C. and noting the time to decolourisation.

Protein Assay

Protein content of samples was estimated by BCA assay (Pierce)

Electrophoresis

Homogeneity of purified proteins was assessed in SDS or native PAGE using a Phastsysytem (Amersham Pharmacia) according to the manufacturer's instructions or in BioRad precast Ready Gels. Visualisation of the separated proteins was achieved using Coomassie Blue (Phastsystem) or silver staining (BioRad) as appropriate.

Micro-Organisms

Organisms were sourced as shown in Table 5.

Amplification & Cloning of DNA

PCR templates were prepared from bacterial colonies or cell pastes using Gene Releaser resin. Typically, a single colony was vortexed for 20 seconds in 28 μl Gene Releaser slurry+28 μl diluent in a 500 μl polypropylene tube and heated for 6 min in a 750W microwave oven at full power. The resin was then pelleted and the supernatant divided into 5 μl aliquots for direct use in PCR reactions. Where DNA of greater purity was required, chromosomal DNA was prepared by standard methods. PCR products intended for cloning and expression were generated using 8U Pfu polymerase, 5 μl native Pfu buffer, 120 nmol⁻¹ forward and reverse primers and 200 μmol⁻¹ of each deoxyribonucleotide in 50 μl total volume in 200 μl thin-wall tubes in a Perkin Elmer PCR9600 thermocycler. Otherwise Taq polymerase was used in place of Pfu. Where necessary, forward primers contained mismatches to introduce restriction enzyme sites through the start codons of amplified genes. Pfu PCR products were ligated into pCR-BluntII-TOPO according to the supplier's instructions and transformed into E. coli. Subsequent DNA manipulation followed standard methods.

Overexpression in E. coli

Genes subcloned into pMTL1015 (Alldread, R. M.; Nicholls, D. J.; Murphy, J. P.; Atkinson, M. A.; Scawen, M. D.; Atkinson, T. & Sundaram, T, K., Escherichia coli malate dehydrogenase gene expression system: characteristics and use as a hyper-expression cassette, unpublished) were expressed in E. coli HMS174.

Overnight cultures of 50 ml were made up to 1 litre with L-broth containing 10 μmol ml⁻¹ tetracycline and grown at 37° C. for a further 24 hours with slow shaking (˜150 rpm) in non-baffled flasks. Genes subcloned into pET21b(+) or pET21d(+) were expressed according to standard methods (4) using E. coli NovaBlue (DE3) as the host. For pET expression aeration was maximized by the use of baffled flasks and anti-foaming agents.

Amplification of nfnB and ywrO Homologues by Reverse PCR

200 ng of StyI-cut or HindIII-cut genomic DNA was self-ligated overnight at 14° C. with 2·5 units T4 DNA ligase in a volume of 50 μl in the buffer supplied with the ligase. Circularized fragments containing the target gene were then amplified by PCR using a pair of outward-facing primers based on a previously cloned 211 bp fragment. After agarose-gel electrophoresis of completed PCRs, ligase-dependent bands of the expected size were excised from the gel and purified

Sequence Analysis

DNA sequencing was performed by CAMR Structural Sciences or Oswel using plasmids eluted in water from Wizard resin, or PCR products eluted in water from PCR clean-up columns. Electropherogram-editing and contig assembly were done using Sequence Manager (LaserGene). Early-release genome databases were searched using the Sanger Centre's gapped-TBLASTA server and the gapped-TBLASTX server at NIH. GenBank was searched using the TFastA program within GCG version 10. Multiple alignments were generated with the PileUp program also within GCG PROGRESS

In vitro Cytotoxicity Tests

Microtitre plates (96 well) were obtained pre-seeded with V79 cells at 10,000 cells/ml (European Collection of Animal Cell Cultures, ECACC) in DMEM+10% foetal calf serum. All treatments were prepared on duplicate plates and transferred to the cells prior to adding enzyme (10 μl) to the appropriate wells. CB 1954 was dissolved in DMSO (Sigma, tissue culture grade) so that the appropriate concentrations could be dispensed by adding 5 μl per well. NAD(P)H was dissolved in sterile PBS to give the appropriate final concentration by adding 10 μl per well. Enzymes were diluted in sterile PBS. All aqueous solutions were filter sterilised before use and operations carried out aseptically in a laminar flow hood. The cells were exposed for 3 h to CB 1954 or SN 23862 (3.9-500 μM in doubling dilutions) alone or in combination with cofactor (NAD(P)H 125 or 250 μM) and enzyme (4 μg) by removing the growth medium and replacing it, after washing twice with PBS, with 200 μl serum free medium containing the various reaction components. After exposure the cells were washed with PBS, fresh medium with serum was added and the plates were left to incubate at 37° C. and 5% CO₂ for 3-4 days until cells in control untreated wells had achieved confluent growth. Cytotoxicity was quantified by sulphorhodamine B (SRB) assay. Briefly, the growth medium was removed and the cells fixed by addition of 100 μl per well of cold 10% TCA for 30 min. The TCA was removed and the fixed cells washed with water before adding 100 μl per well of 0.4% dye in 1% acetic acid and incubating at room temperature for 30 min. Excess dye was removed and the wells washed 4 times with 1% acetic acid. After air drying at room temperature the dye was solubilised by adding 100 μl of 10 mM Tris to each well and shaking for 15 min. The plates were read at 492 nm in a Titertek plate reader. Cytotoxicity towards treated cells was expressed as % of A₄₉₂ of untreated controls and statistical analysis was performed using the Mann-Whitney test. ED₅₀'s were caculated using probit analysis.

Antisera to Recombinant Enzymes

Polyclonal antisera were raised against recombinant enzymes by immunising rabbits with 0.1 μg of protein in 50% PBS:50% Freund's complete adjuvant, total volume 400 μl. Blood was collected by ear bleed after 6-8 weeks and again 10 days after a booster immunisation of protein, and titre assessed by ELISA. 96-well plates were coated with 1 μg ml⁻¹ of the proteins and blocked with 10% v/v foetal calf serum (FCS), antibodies were diluted in assay buffer (1% FCS) and applied in doubling dilutions across the plate from 1 in 100 to 1 in 51 200 or 1 in 150 to 1 in 76 800. A secondary antibody conjugated with HRP (dilution 1 in 10 000) was used to develop the assay with 3,3′,5,5′-tetramethylbenzidine (TMB) as substrate.

Western Blots

Recombinant enzymes were run on SDS PAGE 4-20% gradient at 0.05, 0.5, 5, 50 and 500 ng with 500 ng of the other antigens to test cross-reactivity. The antisera were added at a dilution of 1/10 000 or 1/20 000. Secondary antibody dilutions were as shown above and detection was by ECL (Amersham).

Results and Discussion

Overexpression in E. coli of all Nitroreductase Genes

Of the 15 genes identified, 14 were overexpressed and this work was reported in the annual report for Project 650 in March 2000. Subsequently it was discovered that the enzymes of the thermophile Archaeoglobus fulgidus were in fact relatively inactive, and plans to clone the Thermus thermophilus gene were abandoned.

A total of six recombinant enzymes were selected for further study and, together with the E. coli NfnB enzyme, purified to allow antibody preparation as reported previously (Annual report, project 650, 2000). It has not proved possible to purify the Helicobacter pylori enzyme. However, as this enzyme produces a mixture of the 2 and 4HX with CB 1954, and exhibits relatively low activity, attempts to purify it were abandoned. The Campylobacter jejuni enzyme and the Bacillus subtilis enzymes YrkL, YdeQ and YwrO have now been purified.

C. jejuni NfnB

This enzyme overexpressed in E. coli was purified by anion exchange chromatography using 15 mM piperazine pH 10.0 followed by gel filtration.

YwrO

E. coli bearing pET21 b(+) with ywrO inserted between the EcoR1 and Nde1 sites was grown in 2YT (300 ml+50 μg ml⁻¹ ampicillin). Expression of the YwrO protein was induced by addition of IPTG (200 μg ml⁻¹). The protein was then purified from crude extracts by anion exchange chromatography (FPLC, Mono Q) in 20 mM Tris pH 7.5. Initially, since the substrate specificity of the protein was unknown and it was inactive with either CB 1954 or SN 23862, fractions were identified on the basis of the mobility of the overexpressed protein using SDS-PAGE. Subsequently, active fractions were identified by decolourisation of the azodye, o-methyl red.

YdeQ

YdeQ was purified from a 1.5 ml post-induction (IPTG) lysate by anion exchange chromatography (FPLC Mono Q) in 20 mM Tris pH 7.5. Detection of the presence of the correct protein in fractions eluting from the column on a salt gradient (0-500 mM KCl) was by mobility on SDS-PAGE as for YwrO, since there was no activity with prodrugs.

YrkL

YrkL was purified from crude extracts of an IPTG-induced culture by anion exchange chromatography in 20 mM Tris pH 7.6 and fractions identified by decolourisation of azodye as described for YwrO above

Determination of Polyclonal Antisera Titres by ELISA

Antisera to E. coli B NfnB and six novel proteins (Haemophilus influenzae and Campylobacter jejuni NfnB homologues, YdgI and YodC of Bacillus subtilis, YwrO of B. amyloliquefaciens and Porphyromonas gingivalis YwrO homologue) have been raised in rabbits by inoculation with 100 μg of protein in PBS+ Freund's complete adjuvant. Titres were assessed by ELISA after 8 weeks, and 10 days after a booster inoculation of a further 100 μg of protein per rabbit. Pre-immune sera were also tested.

TABLE 5
Novel nitroreductases, rabbit polyclonal antisera titres

| | Dilution | Secondary | Pre-boost | Post-boost |
|---|---|---|---|---|
| Antiserum | range | Ab dilution | titre (50%) | titre |
| E. coli BNfnB | 150-76800 | 10000 | 1300 | 11000 |
| P. gingivalis YwrO | 50-25600 | 5000 | 1000 | 3100 |
| YwrO BAM | 50-25600 | 5000 | 1200 | 2800 |
| YdgI BS | 150-76800 | 2000 | 4900 | 25000 |
| H. influenzae NfnB | 20-10240 | 10000 | 180 | 760 |
| YodC BS | 100-51200 | 10000 | 3700 | 7500 |
| C. jejuni NfnB | 50-25600 | 5000 | 1100 | 2500 |

96 well plates were coated overnight at 4° C. with the antigens at 1 μg/ml. After washing and blocking antisera were added in doubling dilutions in an appropriate range across the plates. The plates were developed using goat anti-rabbit IgG peroxidase conjugate with TMB as substrate, and read at 450 nm.

Pre-immune sera showed little or no binding to the antigens. Antisera were aliquoted and stored at −20° C.

Western Blots

Antigens were run on SDS PAGE 4-20% gradient at 0.05, 0.5, 5, 50 and 500 ng with 500 ng of other antigens to test cross-reactivity. The antisera were added at a dilution of 1/10000 (α-P. gingivalis YwrO, α-YwrO BAM and α-H. influenzae NfnB) or 1/20000 (α-E. coli B NfnB and α-YdgI). Secondary antibody dilutions were as shown above and detection was by ECL.

Results are shown in FIGS. 3(a-e) E. coli B NfnB could be detected at 5 ng in this system (FIG. 3b) and no cross-reactivity was detected with the other antigens, whereas P. gingivalis YwrO, YwrO BAM and YdgI were detected only at 500 ng, but also with no cross-reactivity. However, antiserum raised against YdgI (FIG. 3d) showed a degree of cross-reactivity with 500 ng of both E. coli B NfnB and YodC, whilst detecting YdgI at 50 ng. Sequence similarity between NfnB and the B. subtilis enzymes is low and the results suggest a greater degree of structural similarity may exist between them.

Kinetics of Each Recombinant Product Against CB1954 and Other Prodrugs

TABLE 6
Novel nitroreductases: physical characterisation and substrate
specificities Prodrug activation

| | CB 1954 | Kₘ | k_{cat} | MW | SN 23862 | | Quinone | Azo- | Flavin |
|---|---|---|---|---|---|---|---|---|---|
| Enzyme | product | CB 1954 | (s⁻¹) | (kDa) | acitvity | Cofactor | reductase | Reductase | reductase |
| C. jejuni | 4HX | 217 | 6.1 | Monomer | 223.7/6.4 | NADPH | Yes | Yes | Yes |
| | | | | 24 | | | | | |
| P. gingivalis | 4HX | 1200 | 3.2 | Dimer | Yes | NADH | No | Yes | No |
| | | | | ˜42 | (weak) | | | | |
| H. influenzae | 4HX | 690 | 56.2 | ˜36 | 3365/39.8 | NADPH | Yes | No | Weak |
| YodC | 4HX | 552.2 | 58.0 | Tetramer | 957.3/53.1 | NADPH | Yes | Yes | Yes |
| | | | | ˜95.5 | | | | | |
| YdgI | 4HX | 3863.9 | 30.3 | Dimer | Yes | NADPH | Yes | Yes | Yes |
| | | | | ˜49 | | | | | |
| H. pylori | 4 > 2HX | ND | ND | Monomer | Weak | NADPH | ND | No | ND |
| | | | | 24 | | | | | |
| Y. pestis | 2HX | ND | ND | Monomer | Weak | NADPH | ND | Yes | ND |
| | | | | 21.3 | | | | | |
| Synechocystis | 2HX | ND | ND | Monomer | Weak | NADPH | ND | No | ND |
| | | | | 22.7 | | | | | |
| A. fulgidus | 4HX | ND | ND | ˜42 | Yes | NADPH | ND | ND | ND |
| 2267 | | | | | | | | | |

Kinetics of the interaction between 5 novel enzymes and the prodrugs CB 1954 and SN 23862 have been estimated (see Table 6). The study was restricted to those enzymes which produce solely the 4HX reduction product of CB 1954 (the nitroreductase of the thermophile, A. fulgidus although purified to homogeneity, proved to have only minimal activity at 37C.)

SN 23862 Activity

Kinetic parameters for SN 23862 were assessed by HPLC assay and determined for YodC BS and 3 NfnB homologues. YdgI BS did not show Michaelis-Menten kinetics, the relationship between [S] and rate of reaction being sigmoidal, suggesting an allosteric interaction. Modelling of the active site region may indicate how this protein differs from the highly related YodC. The crystal structure of NfnB is now available and studies have commenced to model the active sites of YodC, YdgI and H. influenzae NfnB homologue and their interaction with CB 1954 and NADPH. The rate of reduction of SN 23862 shown by P. gingivalis YwrO homologue was too slow for kinetic parameters to be calculated accurately.

Although the SN 23862 Kₘ for YodC is high, the k_{cat} is also high, thus accounting for the cytotoxic action of the combination of enzyme, cofactor and prodrug observed in V79 cells (FIG. 4). Additionally, although kinetic parameters could not be determined for YdgI, it is clear that the cytotoxic derivative of SN 23862 is produced at sufficiently high concentrations for cell killing to occur under the conditions used.

TABLE 7
Substrate and cofactor specificity for YdgI

| Substrate | Cofactor | Kₘ (μM) | k_{cat} (s⁻¹) | k_{cat}/Kₘ |
|---|---|---|---|---|
| Menadione | NADH | 127.0 ± 10 | 628.0 ± 16.8 | 4.94 |
| FMN | NADH | 158.0 ± 16 | 3002.0 ± 94.8 | 19.0 |
| | NADPH | 12.0 ± 1.4 | 345.2 ± 7.6 | 28.7 |
| FAD | NADH | 150.0 ± 19.0 | 2580.7 ± 79 | 17.2 |
| NADH | FMN 1 mM | 59.0 ± 7.0 | 2258.1 ± 64.0 | 38.3 |
| | Menadione | 6.6 ± 8.2 | 766.0 ± 24.0 | 116.1 |
| NADPH | Menadione | 295.0 ± 29 | 96.0 ± 2.8 | 0.3 |
| | 100 μM | | | |

Substrate Specificity

Activity of the B. subtilis enzymes YodC and YdgI with the quinone, menadione and with the flavins FMN and FAD with cofactors NADH and NADPH has been completed and the results are shown in Tables 7 and 8. Assays were carried out spectrophotometrically at 37° C. in 10 mM Tris pH 7.5 using cytochrome c as terminal electron acceptor.

Both these enzymes therefore are flavin reductases and quinone reductases, but in all cases the affinity of YodC for the substrates is higher than that of YdgI. Although they are highly related in amino acid sequence, they differ in their cofactor specificity, YdgI showing a distinct preference for NADH, whereas YodC appears to be more like a DTD, showing similar rates of reaction with either cofactor. Both are potently inhibited by dicumarol (as are DTD and NfnB), but the mechanism of inhibition differs. These results confirm the differences in properties between the two proteins despite their sequence similarity.

Substrate and cofactor specificity for YodC

| Substrate | Cofactor | Kₘ (μM) | k_{cat} (s⁻¹) | k_{cat}/Kₘ |
|---|---|---|---|---|
| Menadione | NADH | 1.0 ± 0.1 | 415.4 ± 14.8 | 415.4 |
| | NADPH | 1.6 ± 0.2 | 329.5 ± 18.4 | 205.9 |
| FMN | NADH | 0.5 ± 0.1 | 293.8 ± 19.7 | 587.6 |
| | NADPH | 1.0 ± 0.1 | 328.2 ± 6.4 | 328.2 |
| FAD | NADH | 0.6 ± 0.1 | 269.0 ± 4.9 | 448.3 |
| | NADPH | 2.4 ± 0.3 | 282.9 ± 7.4 | 117.9 |
| NADH | | | | |
| FMN 5 μM | | 205.0 ± 26.0 | 318.3 ± 11.0 | 1.6 |
| Menadione 5 μM | | 178.0 ± 18.0 | 305.6 ± 21.2 | 1.7 |

The novel enzymes from C. jejuni and H. influenzae were also characterised with respect to the flavins, menadione and cofactors and the results shown in Tables 9 and 10.

TABLE 9
Cofactor and substrate specificity for C. jejuni NfnB

| Substrate | Kₘ | k_{cat} | k_{cat}/Kₘ |
|---|---|---|---|
| Menadione | 1.3 ± 0.2 μM | 66.1 | 50.8 |
| (1 mM NADPH) | | | |
| NADPH | 69.6 ± 8.8 μM | 62.5 | 0.9 |
| (20 μM menadione) | | | |
| FMN | 0.7 ± 0.2 μM | 42.3 | 60.4 |
| FAD | 3.3 ± 0.4 μM | 57.8 | 17.5 |

Both these enzymes show quinone reductase activity with high affinity and distinct preference for NADPH as cofactor. The C. jejuni protein is also a flavin reductase showing high affinity for both FAD and FMN, but H. influenzae NfnB homologue shows little activity with these substrates with either NADH or NADPH as cofactor. Like the B. subtilis enzymes, the former can reduce azodyes, but the latter shows no activity with either o- or p-methyl red in quantitative assays.

TABLE 10
Substrate and cofactor specificity for H. influenzae NfnB

| Substrate | Kₘ | k_{cat} | k_{cat}/Kₘ |
|---|---|---|---|
| Menadione | 9.0 ± 0.6 μM | 177.8 | 19.8 |
| (1 mM NADPH) | | | |
| (1 mM NADH) | 0.8 ± 0.2 μM | 24.1 | 31.0 |
| NADPH | 2.9 ± 0.5 μM | 154.2 | 53.2 |
| (menadione | | | |
| 100 μM) | | | |

Like its homologues in B. subtilis and B. amyloliquefaciens, the YwrO of P. gingivalis is an azoreductase, but it showed little activity with menadione or flavins. Initial studies suggested that it may be an NADH oxidase, but further work is needed to determine its substrate specificity and possible physiological role. It is almost completely inactive with NADPH.

Cytotoxicity Studies Against Cell Lines with Purified Enzymes and Selected Prodrugs

In vitro Cytotoxicity with CB 1954

Enhanced in vitro cytotoxicity against V79 cells of CB 1954 was demonstrated for NfnB, the YwrO of B. amlyoliquefaciens and the 5 other novel proteins. Cytotoxicity was assessed by staining with sulforhodamine B 3-4 days post-treatment with prodrug, enzyme and cofactor. The H. influenzae NfnB homologue was the most potent, whilst the YwrO homologues were the least potent of the novel enzymes. (FIG. 3)

In vitro Cytotoxicity (SN 23862)

In vitro cytotoxicity assays were carried out using this alternative prodrug with NfnB and five of the novel enzymes (YwrO of B. amyloliquefaciens is inactive with this substrate) (FIG. 4). Dose-related cytotoxicity was seen with all the enzymes except for P. gingivalis YwrO homologue. The order of potency was similar to that with CB 1954 the most potent being the NfnB homologue of H. influenzae. Kinetic studies with P. gingivalis YwrO homologue and SN 23862 indicate that the rate of reduction is very slow (see above) and this may explain the lack of activity in cytotoxicity assays where a critical level of the cytotoxin is probably necessary for effective cell killing.

TABLE 11
ED₅₀'s for novel nitroreductases in in vitro cytotoxicity tests
using CB 1954 or SN 23862, calculated by probit analysis

| Enzyme | ED₅₀ CB 1954 (μM) | ED₅₀ SN 23862 |
|---|---|---|
| YwrO BAM | 137.1 | — |
| YdgI | 15.3 | 76.6 |
| YodC | 20.3 | 74.2 |
| NfnB | 6.3 | 30.7 |
| H. influenzae NfnB | 4.7 | 17.1 |
| C. jejuni NfnB | 55.8 | 102.3 |
| P. gingivalis YwrO | 252.3 | — |

Pae3

The protein encoded by Pae3 was overexpressed in E. coli and purified by anion exchange chromatography. It is most highly related to the human form of DTD (cf other nitroreductase sequences used to search databases) and in this context, it is perhaps not surprising that it is inactive with both prodrugs, CB 1954 and SN 23862. More unexpectedly, it is also inactive with flavins and azodyes, properties which are shared by several members of the DTD/YwrO family of enzymes. However, it is a quinone reductase, and kinetic parameters were determined for this substrate using NADPH as cofactor, the rate of reaction using NADH as cofactor being approximately 5-fold lower.

TABLE 12
Kinetic parameters for Pae3 of Pseudomonas aeruginosa

| | Kₘ | k_{cat} | Kₛᵢ |
|---|---|---|---|
| Menadione | 3.8 μM | 153.4 s⁻¹ | 18.0 μM |
| NADPH | 308.4 μM | 33.8 s⁻¹ | — |

Efa1

This gene is more highly related in sequence to ydgI compared to the other sequences used to search databases. The gene product of Efa1 expressed in E. coli was purified by anion exchange chromatography in 20 mM Tris pH 7.6 and its substrate specificity determined using the prodrugs, menadione and flavins (Table 13). The reduction of CB 1954 resulted in the formation of both the 2 and 4HX products, in similar proportions to those formed by NfnB (approximately 50% of each product formed). SN 23862 reduction formed the 2HX cytotoxic product, but kinetic parameters were not determined for this substrate. It is a flavin, azo- and quinone reductase and shows a distinct preference for NADH as cofactor. Despite the sequence similarity to YdgI, therefore, the properties of the protein differ significantly (cofactor specificity, product formation) indicating substantial differences in structure.

TABLE 13
Kinetic parameters for Efa 1 of Enterococcus faecalis

| Substrate | Kₘ | k_{cat} | Kₛᵢ |
|---|---|---|---|
| CB 1954 | 4100 μM | 12.0 s⁻¹ | |
| Active | ND | ND | — |
| Menadione | 107.4 μM | 264.8 s⁻¹ | 18.0 μM |
| NADH (1 mM | 44.3 μM | 314.9 s⁻¹ | — |
| FMN) | | | |
| FMN | 104.3 μM | 340.0 s⁻¹ | — |
| FAD | 133.8 μM | 187.8 s⁻¹ | — |

Smu2

The Smu2 gene shows sequence similarity to the nfnB homologue of H. influenzae. The protein, overexpressed in E. coli Top 10 was purified from a crude extract by anion exchange chromatography in 15 mM piperazine pH 10.0 (pI estimated from sequence to be 8.36). Like the “parent” protein it rapidly reduces CB 1954 with formation of the 4HX product only and it uses NADPH preferentially as cofactor. The cytotoxic 2HX product was formed on reduction of SN 23862. With menadione as substrate, it was virtually inactive with NADH. It is a potent quinone reductase but shows no activity with flavins, again resembling H. influenzae NfnB.

TABLE 14
Kinetic parameters for Smu2 of Streptococcus mutans

| | Kₘ | k_{cat} |
|---|---|---|
| CB 1954 | 2700 μM | 96.4 s⁻¹ |
| Menadione | 2.7 μM | 201.0 s⁻¹ |
| NADPH | 1.3 μM | 188.7 s⁻¹ |
| (100 uM menadione) | | |

Pmu2

This gene also shows sequence similarity to the nfnB of H. influenzae and, like Smu2 shares similar substrate and cofactor specificity. It is a quinone reductase with high affinity and uses NADPH as cofactor, however it can use NADH but with a 2-fold decrease in rate of reaction (substrate menadione). It has little activity with flavins with either cofactor. It forms the 4HX reduction product of CB 1954 exclusively and has a greater affinity for this substrate than Smu2. With SN 23862 it forms the cytotoxic 2HX product, but kinetic parameters were not determined.

TABLE 15
Kinetic parameters for Pmu2 of Pasturella multocida

| | Kₘ | k_{cat} |
|---|---|---|
| CB 1954 | 692.4 μM | 8.6 s⁻¹ |
| Menadione | 2.6 μM | 23.5 s⁻¹ |
| NADPH | 2.9 μM | 24.6 s⁻¹ |
| (25 uM menadione) | | |

References

1. Anlezark, G. M., R. G. Melton, R. F. Sherwood, B. Coles, F. Friedlos, and R. J. Knox. 1992. The bioactivation of 5-(aziridin-1-yl)-2,4-dinitrobenzamide (CB1954)—I. Purification and properties of a nitroreductase enzyme from Escherichia coli—a potential enzyme for antibody-directed enzyme prodrug therapy (ADEPT). Biochem Pharmacol. 44(12):2289-95.

2. Anlezark, G. M., R. G. Melton, R. F. Sherwood, W. R. Wilson, W. A. Denny, B. D. Palmer, R. J. Knox, F. Friedlos, and A. Williams. 1995. Bioactivation of dinitrobenzamide mustards by an E. coli B nitroreductase. Biochem Pharmacol. 50(5):609-18.

3. Knox, R. J., M. P. Boland, F. Friedlos, B. Coles, C. Southan, and J. J. Roberts. 1988. The nitroreductase enzyme in Walker cells that activates 5-(aziridin-1-yl)-2,4-dinitrobenzamide (CB 1954) to 5-(aziridin-1-yl)-4-hydroxylamino-2-nitrobenzamide is a form of NAD(P)H dehydrogenase (quinone) (EC 1.6.99.2). Biochem Pharmacol. 37(24):4671-7.

4. Studier F W, R. A. H., Dunn J J, Dubendorff J W. 1990. Use of T7 polymerase to direct expression of cloned genes. Meth Enzymol. 185:60-85.

5. Zenno, S., T. Kobori, M. Tanokura, and K. Saigo. 1998. Conversion of NfsA, the major Escherichia coli nitroreductase, to a flavin reductase with an activity similar to that of Frp, a flavin reductase in Vibrio harveyi, by a single amino acid substitution. J Bacteriol. 180(2):422-5.

6. Zenno, S., H. Koike, M. Tanokura, and K. Saigo. 1996. Conversion of NfsB, a minor Escherichia coli nitroreductase, to a flavin reductase similar in biochemical properties to FRase I, the major flavin reductase in Vibrio fischeri, by a single amino acid substitution. J Bacteriol. 178(15):4731-3.

The invention thus provides nitroreductase enzymes, DNA and genes therefor and methods of obtaining such enzymes and of using the enzymes and DNA coding therefor in clinical applications.

TABLE 1
Characteristics of nitroreductase enzymes from Bacillus amyloliquefaciens

| | M. Wt | CB1954 | | | SN23862 | |
|---|---|---|---|---|---|---|
| ENZYME | (Kda) | Product | Km | kcat | Km | kcat |
| E. coli NCnB | 24 | 2/4HX | 862 | 6.0 | 2500 | 26.4 |
| Rat DTD | 33 | 4HX | 826 | 0.07 | inactive | inactive |
| Bam YwrO | 22 | 4HX | 617 | 2.0 | inactive | inactive |

NfnB—nitroreductase of E. coli B; DTD—DT Diaphorase;, and; Bam YwrO—cloned Bacillus amyloliquefaciens nitroreductase

TABLE 2
Characteristics of nitroreductase enzymes found in the
Bacillus subtilis genome

| | DTD-like Family | | | NfnB-like Family | |
|---|---|---|---|---|---|
| | YwrO | YrkL | YdeQ | YdgI | YodC |
| Homology^{a} | 70% | 54% | 51% | 25% | 24% |
| CB1954 | 4HX | inactive | inactive | 2/4HX | 2/4HX |
| SN23862 | inactive | inactive | inactive | active | active |

^{a}DTD-like family homologies are to the Bacillus amyloliquefaciens YwrO, NfnB-like family homologies are to the E.coli B nitroreductase.

TABLE 3
Fractionation of nitroreductase activity in cell
extracts of Bacillus lautus and Bacillus pumilis

| | | | |
|---|---|---|---|
| ENZYME | M. Wt | CB1954 | SN23862 |

| ACTIVITY | (kDa) | Product | Km | Km |
|---|---|---|---|---|
| B. pumilis CP044 | | | | |
| Peak 1 | ND | 4HX | v. low | ND |
| Peak 2 | ND | 4HX | >1000 | ND |
| Peak 3 | ND | 2/4HX | 999 | ND |
| B. lautus CP060 | | | | |
| Peak 1 | 35 | 2HX | 211 | 325 |
| Peak 2 | 42 | 4HX | >2000 | none |
| Peak 3 | 47 | 4HX | 257 | active |

TABLE 4
Characteristics of nitroreductase activity of thermophiles
identified as being sensitive to CB 1954

| | CB1954 | | | SN23862 | |
|---|---|---|---|---|---|
| STRAIN | Product | NADH | NADPH | NADH | NADPH |
| 1078 | 2/4HX | 13.8 | 22.6 | 8.5 | 17.6 |
| 2122^{a} | 2/4HX | 36.6 | 56.0 | 33.4 | 62.8 |
| 6012^{b} | 4 > 2HX | 15.2 | 37.8 | 8.2 | 35.2 |
| 6013^{c} | 2HX | 9.8 | 49.4 | 6.4 | 39.0 |
| 6031^{d} | 2HX | 11.9 | 42.1 | 8.2 | 33.8 |
| 6036 | 2HX | 10.7 | 26.7 | 7.3 | 26.2 |
| 6044 | 2HX | 4.0 | 21.3 | 4.5 | 9.9 |

[Identified as Bacillus thermoflavus ^{a}, Bacillus licheniformis^{b}, Bacillus licheniformis^{c}, Bacillus alkophilus^{d}]

1. A nucleic acid comprising
(a) a DNA encoding a nitroreductase which preferentially reduces CB1954 to a cytotoxic 4-hydroxylamine (4HX) derivative instead of a non-cytotoxic 2-hydroxylamine derivative, operatively coupled to
(b) a promoter for expression of the DNA,
wherein the nucleic acid is selected from the group consisting of SEQ ID NO:s 10, 12,17, 20, 21, 23, 25 and 29.

2. A viral vector, comprising:—
(a) a DNA encoding a nitroreductase which preferentially reduces CB1 954 to a cytotoxic 4-hydroxylamine (4HX) derivative instead of a non-cytotoxic 2-hydroxylamine derivative, operatively coupled to
(b) a promoter for expression of the DNA,
wherein the nucleic acid is selected from the group consisting of SEQ ID NO:s 10, 12, 17, 20, 21,23, 25 and 29.

3. A method of preparing a nitroreductase, comprising expressing a gene in a bacterial cell, wherein the gene comprises a nucleic acid selected from the group consisting of SEQ ID NO:s 10, 12, 17, 20, 21,23, 25 and 29.

4. A nucleic acid comprising—
(a) a DNA encoding a nitroreductase which preferentially reduces CB1954 to a cytotoxic 4-hydroxylamine (4HX) derivative instead of a non-cytotoxic 2-hydroxylamine derivative, operatively coupled to
(b) a promoter for expression of the DNA.

5. A viral vector, comprising:—
(a) a DNA encoding a nitroreductase which preferentially reduces CB1954 to a cytotoxic 4-hydroxylamine (4HX) derivative instead of a non-cytotoxic 2-hydroxylamine derivative, operatively coupled to
(b) a promoter for expression of the DNA.

6. A method of preparing a nitroreductase, comprising expressing a gene in a bacterial cell, wherein the gene encodes a nitroreductase which preferentially reduces CB1954 to a cytotoxic 4-hydroxylamine (4HX) derivative instead of a non-cytotoxic 2-hydroxylamine derivative.

7. The method of claim 3, further comprising combining the nitroreductase with a pharmaceutically acceptable carrier.

8. The method of claim 6, further comprising combining the nitroreductase with a pharmaceutically acceptable carrier.


SEQUENCE LISTING (29 sequences)

SEQ ID NO: 1 - DNA, 525 bp, Bacillus amyloliquefaciens
  CDS (1)..(525)
gtgaaagtattggtattagcggttcaccctgacatggagaactcagcggtcaataaggcatgggcagaagaattaaaaaaacatgatgaactcacggtccgtgagctttataaagaatatccggacgggcaaatcgatgcggaaaaggaacgtcagctgtgtgaacagtatgaccggatcgtatttcaatttccgctgtattggtacagtgcgcctccgcttttaaaaacatggatggatcatgtgctgtcgtacggctgggcctacggctccaaaggaaaggcgctgcatggcaaagaattgatgctggctgtttccgtaggtgccggagaggatgcataccaggcaggagggtcaaaccactttacattgagcgagctgttaaggccgtttcaggcaatggctaattttacaggtatgacctatttgccggctttcgcgctgtacggtgtaaatggggcggatgcgacggatattcatgacaatgccaaacgtctggctgcttacataaagaaatcattttaa

SEQ ID NO: 2 - protein, 174 aa, Bacillus amyloliquefaciens
VKVLVLAVHPDMENSAVNKAWAEELKKHDELTVRELYKEYPDGQIDAEKERQLCEQYDRIVFQFPLYWYSAPPLLKTWMDHVLSYGWAYGSKGKALHGKELMLAVSVGAGEDAYQAGGSNHFTLSELLRPFQAMANFTGMTYLPAFALYGVNGADATDIHDNAKRLAAYIKKSF

SEQ ID NO: 3 - DNA, 528 bp, Bacillus subtilis
  CDS (1)..(528)
atgaaaatattggttttggcagtgcatcctcatatggagacctcagttgttaataaggcgtgggctgaggaattgagtaaacatgacaatatcacagtacgggatctttataaggaatacccggatgaagcgatagatgttgcgaaggaacagcagctgtgcgaggaatacgatcggattgtctttcaattcccgctatattggtacagctctccgccgctcttgaaaaaatggcaggatcttgtgctgacttatggctgggcttttggttcagaaggaaatgccttgcatggcaaggagctgatgctggctgtatcaacagggagcgaagcagaaaaatatcaagcgggcggagcaaatcattactcgatcagtgagctattgaaaccatttcaggccacgagtaatctgatcggcatgaagtatctgcctccatatgtgttctatggcgtgaattatgcagctgcagaggatatttctcacagtgcaaaacggttagccgaatacatccagcagccttttgtttaa

SEQ ID NO: 4 - protein, 175 aa, Bacillus subtilis
MKILVLAVHPHMETSVVNKAWAEELSKHDNITVRDLYKEYPDEAIDVAKEQQLCEEYDRIVFQFPLYWYSSPPLLKKWQDLVLTYGWAFGSEGNALHGKELMLAVSTGSEAEKYQAGGANHYSISELLKPFQATSNLIGMKYLPPYVFYGVNYAAAEDISHSAKRLAEYIQQPFV

SEQ ID NO: 5 - DNA, 525 bp, Bacillus subtilis
  CDS (1)..(525)
atgaaaacattagttatcgttatacatcctaatttggaaacgtctgttgtcaacaaaacctggatgaatcgtttaaagcaagagaaagacattacggttcatgacctgtacggtgaataccctaattttatcattgatgtagaaaaagagcagcagctcctgttagatcatgagcgtatcgtttttcagttcccaatgtattggtacagcagtcccgcgttactcaaacaatgggaagatgatgtgttaacacatggctgggcttatggaactggaggaactaaattgcatggaaaagaactactcttagctatctcctcaggcgcacaggaatctgattatcaagcaggcggagaatataatatcacgatcagcgagcttatcagaccgtttcaagtcactgctaactatataggaatgcgttttcttcctgcgtttacacaatatgggacacttcatctttcaaaagaagatgttaagaacagtgcggagagattggttgactatcttaaagccgagcattaa

SEQ ID NO: 6 - protein, 174 aa, Bacillus subtilis
MKTLVIVIHPNLETSVVNKTWMNRLKQEKDITVHDLYGEYPNFIIDVEKEQQLLLDHERIVFQFPMYWYSSPALLKQWEDDVLTHGWAYGTGGTKLHGKELLLAISSGAQESDYQAGGEYNITISELIRPFQVTANYIGMRFLPAFTQYGTLHLSKEDVKNSAERLVDYLKAEH

SEQ ID NO: 7 - DNA, 594 bp, Bacillus subtilis
  CDS (1)..(594)
atggatcatatgaaaacactcgtactcgttgtacatccgaatatagaatcctctcgtatcaataaaaagtggaaagaagccgttttaagtgaaccagatgtaactgtccatgatctttatgaaaaatatcgcgatcaaccaattgatgtggaatttgaacaacagcagctcctggcccatgaccgtatcgtttttcagtttccattatactggtacagcagcccaccgcttttaaaacagtggtttgatgaagtgtttacgtttggctgggctcatggtcccggcggaaataaattgaaggggaaagagtgggtaactgccatgtccatcggttcacctgaacactcttatcaagccggcggatataacttgttttcgataagcgagctgacaaaaccgttccaagcatctgcccatttagtaggcatgacctatctgccttcctttgccgaatatcgcgccaatacaatcagtgaccaagaaattgccgaaagtgcgaatcggtatgtaaagcatattacaaatatagaattaaacccgaaggttcgcctgcaaaggtatttgaaacagctggagagtgtcgatttaacataa

SEQ ID NO: 8 - protein, 197 aa, Bacillus subtilis
MDHMKTLVLVVHPNIESSRINKKWKEAVLSEPDVTVHDLYEKYRDQPIDVEFEQQQLLAHDRIVFQFPLYWYSSPPLLKQWFDEVFTFGWAHGPGGNKLKGKEWVTAMSIGSPEHSYQAGGYNLFSISELTKPFQASAHLVGMTYLPSFAEYRANTISDQEIAESANRYVKHITNIELNPKVRLQRYLKQLESVDLT

SEQ ID NO: 9 - DNA, 630 bp, Bacillus subtilis
  CDS (1)..(630)
atgatcaaaacaaacgattttatggaaattatgaaaggccgccgttctatccgcaactatgatccggcagtaaaaatcagcaaagaagaaatgacagagatcttagaggaagcaacaactgccccatcttctgttaacgcgcagccatggcgttttcttgtcattgacagcccggaaggaaaagaaaagctcgcaccgcttgcaagctttaaccaaacacaagtcacaacatcatctgctgtcatcgctgtatttgcagacatgaacaacgcagactatctagaagaaatctattcaaaagccgtggaacttggttacatgccgcaggaggtcaaagacagacaaatcgccgcgctgaccgcacattttgaaaagcttccggcacaggtcaaccgtgaaacgatcctgattgacggaggtcttgtttccatgcagctgatgctgactgcacgcgcgcatggctacgatacaaacccgatcggcggatacgataaagaaaacatcgcggaaaccttcggattagataaagaacgttatgtaccggttatgctactttctatcggaaaagcagcagacgaaggctatgcttcctaccgtctgccgattgatacaattgcagaatggaaataa

SEQ ID NO: 10 - protein, 209 aa, Bacillus subtilis
MIKTNDFMEIMKGRRSIRNYDPAVKISKEEMTEILEEATTAPSSVNAQPWRFLVIDSPEGKEKLAPLASFNQTQVTTSSAVIAVFADMNNADYLEEIYSKAVELGYMPQEVKDRQIAALTAHFEKLPAQVNRETILIDGGLVSMQLMLTARAHGYDTNPIGGYDKENIAETFGLDKERYVPVMLLSIGKAADEGYASYRLPIDTIAEWK

SEQ ID NO: 11 - DNA, 609 bp, Bacillus subtilis
  CDS (1)..(609)
atgacgaatactctggatgttttaaaagcacgtgcatctgtaaaggaatatgatacaaatgccccgatctctaaggaggagctgactgagctattagaccttgccactaaagcgccttctgcttggaaccttcagcattggcattttacagtattccacagcgatgaatcaaaagcggagcttcttcctgtagcgtataatcaaaaacaaatcgttgagtcttctgctgttgttgccattttaggcgatttaaaggcaaatgaaaacggtgaagaagtttatgctgaattagcaagccaaggctatattacggatgaaatcaaacaaacattgctcggccaaatcaacggtgcttaccaaagcgagcaattcgcacgtgattccgctttcttaaatgcttctttagctgctatgcagcttatgattgccgcaaaagcaaaaggttatgacacttgcgcaatcggcggatttaacaaagagcagttccaaaagcaatttgatatcagtgagcgctatgttccggttatgcttatttcaatcggcaaagcagtgaagcctgcgcatcaaagcaaccgtctgccgctttcaaaagtatcaacttggctgtaa

SEQ ID NO: 12 - protein, 202 aa, Bacillus subtilis
MTNTLDVLKARASVKEYDTNAPISKEELTELLDLATKAPSAWNLQHWHFTVFHSDESKAELLPVAYNQKQIVESSAVVAILGDLKANENGEEVYAELASQGYITDEIKQTLLGQINGAYQSEQFARDSAFLNASLAAMQLMIAAKAKGYDTCAIGGFNKEQFQKQFDISERYVPVMLISIGKAVKPAHQSNRLPLSKVSTWL

SEQ ID NO: 13 - DNA, 555 bp, Escherichia coli
  CDS (1)..(555)
atgatgtctcagccagcgaaagttttgctgctgtatgcccatccggaatctcaggactcggtggcaaaccgggtactgcttaaaccggccacgcagctcagcaatgttaccgtgcacgacctttacgcgcactatcccgatttttttattgatatcccccgtgagcaggcattactgcgcgagcacgaggtgattgtctttcagcatcctctttatacctatagctgcccggcgctactgaaagagtggctggaccgggtattaagtcgtggttttgccagcgggccgggaggaaaccaactggcgggaaagtactggcgtagcgtgattaccaccggcgagccggaaagtgcttaccgttatgacgcgctgaatcgctacccgatgagcgatgtgctgcgcccctttgaactggcggcgggcatgtgccggatgcattggttaagtcccatcattatttactgggcgagacggcaaagcgcacaggagctggcgagccacgccagagcctacggtgactggctggcaaatccgctgtctccaggaggccgctga

SEQ ID NO: 14 - protein, 184 aa, Escherichia coli
MMSQPAKVLLLYAHPESQDSVANRVLLKPATQLSNVTVHDLYAHYPDFFIDIPREQALLREHEVIVFQHPLYTYSCPALLKEWLDRVLSRGFASGPGGNQLAGKYWRSVITTGEPESAYRYDALNRYPMSDVLRPFELAAGMCRMHWLSPIIIYWARRQSAQELASHARAYGDWLANPLSPGGR

SEQ ID NO: 15 - DNA, 531 bp, Escherichia coli
  CDS (1)..(531)
atgattcttataatttatgcgcatccgtatccgcatcattcccatgcgaataaacggatgcttgaacaggcaaggacgctggaaggcgtcgaaattcgctctctttatcaactctatcctgacttcaatatcgatattgccgccgagcaggaggcgctgtctcgcgccgatctgatcgtctggcagcatccgatgcagtggtacagcattcctccgctcctcaaactttggatcgataaagttttctcccacggctgggcttacggtcatggcggcacggcgctgcatggcaaacatttgctgtgggcggtgacgaccggcggcggggaaagccattttgaaattggtgcgcatccgggctttgatgtgctgtcgcagccgctacaggcgacggcaatctactgcgggctgaactggctgccaccgtttgccatgcactgcacctttatttgtgacgacgaaaccctcgaagggcaggcgcgtcactataagcaacgtctgctggaatggcaggaggcccatcatggatag

SEQ ID NO: 16 - protein, 176 aa, Escherichia coli [sequence omitted]

SEQ ID NO: 17 - protein, 220 aa, Haemophilus influenzae
MTQLTREQVLELFHQRSSTRYYDPTKKISDEDFECILECGRLSPSSVGSEPWKFLVIQNKTLREKMKPFSWGMINQLDNCSHLVVILAKKNARYDSPFFVDVMARKGLNAEQQQAALTKYKALQEEDMKLLENDRTLFDWCSKQTYIALANMLTGASALGIDSCPIEGFHYDKMNECLAEEGLFDPQEYAVSVAATFGYRSRDIAKKSRKGLDEVVKWVG

SEQ ID NO: 18 - protein, 205 aa, Thermus aquaticus
MEATLPVLDAKTAALKRRSIRRYRKDPVPEGLLREILEAALRAPSAWNLQPWRIVVVRDPATKRALREAAFGQAHVEEAPVVLVLYADLEDALAHLDEVIHPGVQGERREAQKQAIQRAFAAMGQEARKAWASGQSYILLGYLLLLLEAYGLGSVPMLGFDPERVRAILGLPSRAAIPALVALGYPAEEGYPSHRLPLERVVLWR

SEQ ID NO: 19 - protein, 200 aa, Synechocystis PCC6803
MDTFDAIYQRRSVKHFDPDHRLTAEEERKLHEAAIQAPTSFNIQLWRFLIIRDPQLRQTIREKYGNQAQMTDASLLILVAADVNAWDKDPARYWRNAPREVANYLVGAIAFYGGKPQLQRDEAQRSIGMAMQNLMLAAKAMGYDSCPMIGFDLQKVAELVKLPADYAIGPMVAIGKRTEDARAKGGQTPLEELVWENSFA

SEQ ID NO: 20 - protein, 172 aa, Archaeoglobus fulgidus
MECLDLLFRRVSIRKFTQDDVDDEILMKILEAGNAAPSAGNLQARDFVVIRNPETKKRLAMAALKQMFIAEAPVVIVVCANYPRSMRVYGERGRLYAEQDATAAIENILLAVTALNLGAVWVGAFDEEQVSEILELPEYVRPMAIIPIGHPAENPSPRNRYPVSMLTHFEKW

SEQ ID NO: 21 - protein, 174 aa, Archaeoglobus fulgidus
MEECLKMIYTRRSIRVYSDRQISDEDIEKILKAAMLAPSAGNEQPWHFIVVRDREMLKKMSEAFTFGQMLPNASAAIVVCADPKLSKYPYDMWVQDCSAATENILLAARCLGIGSVWLGVYPREERMKALRELLGIPENIVVFSVVSLGYPKDEKDFYEADDRFNPDRIHREKW

SEQ ID NO: 22 - DNA, 606 bp, Campylobacter jejuni
  CDS (1)..(606)
atgaaaaaagaacttgaaatttttagcacaagatattcttgtagaaattttaaaaatgaaaaactcaaaaaagaggatttaaattctattttagaaatagcaagattaagccccagttccttgggactggaaccttggaaatttatagtagtgcaagatgagaaaagaaaagaagaactttctaaaatttgcaatcaacaaaaacatgtaaaagattgtgctgcattaattataatcatttcaagacttgattttttggattattttgaagaaaaacttagaaaaagagatatgagtgaaacagaaatgcaaaaacgcttagatacttatatgccttttttaaaatctctaaatcaagaacaaaaaatatcttatgcaagagaacaagctcatatagctctagctagcatactttacagtgctaatgctttaaatatagcaagctgcactataggtggttttgataaagaaaagcttgattcttatttatcacttgatattcaaaaagaaagatcaagtttggtggtggctttaggatattgcaacgataaaaaaaatcctcaaaaaaatcgttttagttttgatgaagttgtaaaatttatttaa

SEQ ID NO: 23 - protein, 201 aa, Campylobacter jejuni
MKKELEIFSTRYSCRNFKNEKLKKEDLNSILEIARLSPSSLGLEPWKFIVVQDEKRKEELSKICNQQKHVKDCAALIIIISRLDFLDYFEEKLRKRDMSETEMQKRLDTYMPFLKSLNQEQKISYAREQAHIALASILYSANALNIASCTIGGFDKEKLDSYLSLDIQKERSSLVVALGYCNDKKNPQKNRFSFDEVVKFI

SEQ ID NO: 24 - DNA, 522 bp, Porphyromonas gingivalis
  CDS (1)..(522)
atgaaaaaaacgctcgtaatagtcgttcaccccgatttgaccaaatccgttatcaacaaggcttgggccaaagccatcgaaggtgcagccactatccaccatctctacgaacagtatccgaacggacaaatcgatctagcacatgaacaagccctgctggaggctcatgaccgcatcgtcttccaattccccctctattggtatgcagctccctatctgctgaagaagtggatggacgaggtctttactgagggctgggcctatggtgccggtggagacaagatggagggtaaagaaatctgtgcagcagtctcctgcggatcacccaaatcagcttttgccgaaggagcacagcaatgccacacgctgcgaagctacttgaatgtattcgacgggatagctgctttcctgcgcgctcgattcaccggctaccatgcctgctacgattcctacaatcctcgcctgccggaaatgctgccggccaactgcgaagcctatctccgctttatcaaaggagaatga

SEQ ID NO: 25 - protein, 173 aa, Porphyromonas gingivalis
MKKTLVIVVHPDLTKSVINKAWAKAIEGAATIHHLYEQYPNGQIDLAHEQALLEAHDRIVFQFPLYWYAAPYLLKKWMDEVFTEGWAYGAGGDKMEGKEICAAVSCGSPKSAFAEGAQQCHTLRSYLNVFDGIAAFLRARFTGYHACYDSYNPRLPEMLPANCEAYLRFIKGE

SEQ ID NO: 26 - DNA, 552 bp, Yersinia pestis
  CDS (1)..(552)
atgatgttgcagccgccgaaggttttgctgctgtatgcccatccggaatcacaggactcggtcgctaaccgggttttactgcaaccggtacagcagttagaacatgtcactgtgcacgatctttatgcacattatccggatttctttattgatattcatcatgagcagcaattgctacgtgatcatcaagttattgtatttcaacatcctttatatacttacagttgccctgcattactgaaagagtggttggatcgggtactggcacgtggtttcgccaatggcgttggcggccatgcactgacgggaaagcactggcgctcggtgattaccaccggtgagcaggagggaacttaccgtattgggggatataaccgttacccaatggaagacattctgcgtcctttcgaattgacggcggctatgtgccatatgcattggattaatccgatgattatttactgggccagacgccaaaagccggaaacactcgccagtcacgcacaagcttatgtgcaatggctgcagtcaccgctcacgagaggactctga

SEQ ID NO: 27 - protein, 183 aa, Yersinia pestis
MMLQPPKVLLLYAHPESQDSVANRVLLQPVQQLEHVTVHDLYAHYPDFFIDIHHEQQLLRDHQVIVFQHPLYTYSCPALLKEWLDRVLARGFANGVGGHALTGKHWRSVITTGEQEGTYRIGGYNRYPMEDILRPFELTAAMCHMHWINPMIIYWARRQKPETLASHAQAYVQWLQSPLTRGL

SEQ ID NO: 28 - DNA, 633 bp, Helicobacter pylori
atgaaatttttggatcaagaaaaaagaagacaattgctaaacgagcgccattcttgcaagatgttcgacagccattatgagttttctagtgaagaattagaagaaatcgctgaaatcgctaggctatcgccaagctcttacaacacgcagccatggcattttgtgatggttactaataaggatttaaaaaaacaaattgcagcgcacagctattttaatgaagaaatgattaaaagcgcttcagcgttaatggtggtatgctctttaaaacccagcgagttgttacccactggccactacatgcaaaacctttacccggagtcttataaggttagagtgatcccctcttttgctcaaatgcttggcgtgagattcaaccacagcatgcaaaaattagaaagctatattttggagcaatgctatatcgctgtggggcaaatttgcatgggcgtgagcttaatgggattggatagttgcattattggaggctttgatcctttaaaagtgggcgaagttttagaagagcgtatcaataaacctaaaatcgcatgcttgatcgctttgggcaagagggtggcagaagcgagccaaaaatcaagaaaatcaaaagttgatgccattacttggttgtga

SEQ ID NO: 29 - protein, 210 aa, Helicobacter pylori
MKFLDQEKRRQLLNERHSCKMFDSHYEFSSEELEEIAEIARLSPSSYNTQPWHFVMVTNKDLKKQIAAHSYFNEEMIKSASALMVVCSLKPSELLPTGHYMQNLYPESYKVRVIPSFAQMLGVRFNHSMQKLESYILEQCYIAVGQICMGVSLMGLDSCIIGGFDPLKVGEVLEERINKPKIACLIALGKRVAEASQKSRKSKVDAITWL